SEQUENCE LISTING (6 sequences)

SEQ ID NO: 1 - DNA, 19 bp, Artificial sequence
  Single strand DNA oligonucleotide
cgacagtcagccgcatctt

SEQ ID NO: 2 - DNA, 21 bp, Artificial sequence
  Single strand DNA oligonucleotide
ccaatacgaccaaatccgttg

SEQ ID NO: 3 - DNA, 22 bp, Artificial sequence
  Single strand DNA oligonucleotide
ggatggtcaaagaagtggttcg

SEQ ID NO: 4 - DNA, 19 bp, Artificial sequence
  Single strand DNA oligonucleotide
cctgtgggctcttcggttt

SEQ ID NO: 5 - DNA, 22 bp, Artificial sequence
  Single strand DNA oligonucleotide
cgaggactctggacagtagagg

SEQ ID NO: 6 - DNA, 22 bp, Artificial sequence
  Single strand DNA oligonucleotide
gatcttgagccccagttttctg

(19) United States
(12) Patent Application Publication
(10) Pub. No.: US 2009/0257987 A1
(43) Pub. Date: Oct. 15, 2009

(54) Method of generating dopamine-secreting cells

(75) Inventors: Daniel Offen, Kfar HaRoe (IL); Eldad Melamed, Tel-Aviv (IL); Ran Barzilay, Tel-Aviv (IL); Inna Kan, Rechovot (IL)

Correspondence Address: MARTIN D. MOYNIHAN d/b/a PRTSI, INC., P.O. BOX 16446, ARLINGTON, VA, 22215, US

(73) Assignee: Ramot At Tel Aviv University Ltd., Tel-Aviv (IL)

(21) Appl. No.: 12/385,460

(22) Filed: Apr. 8, 2009

Related U.S. Application Data
(60) Provisional application No. 61/071,054, filed on Apr. 10, 2008.

(51) Int. Cl. A61K 45/00; C12N 5/08; A61P 25/28

(52) U.S. Cl. 424/93.7; 435/377; 435/366

(57) ABSTRACT

Methods of generating cells which secrete dopamine are disclosed. One method comprises incubating mesenchymal stem cells in a culture medium comprising brain derived neurotrophic factor (BDNF), wherein the culture medium comprises no more than 0.1 μM retinoic acid (RA). Another method comprises incubating mesenchymal stem cells in a culture medium comprising glial-derived neurotrophic factor GDNF, transforming growth factor β3 (TGFβ3) and retinoic acid (RA). Cells generated by the above-described methods are also disclosed as well as uses thereof.

### RELATED APPLICATIONS

This application claims the benefit of U.S. Provisional Patent Application No. 61/071,054, filed on Apr. 10, 2008, the contents of which is incorporated herein by reference as if fully set forth herein.

### FIELD AND BACKGROUND OF THE INVENTION

The present invention, in some embodiments thereof, relates to methods of generating cells which secrete dopamine and uses thereof.

Parkinson's disease is an age-related disorder characterized by progressive loss of dopamine producing neurons in the substantia nigra of the midbrain, which in turn leads to progressive loss of motor functions manifested through symptoms such as tremor, rigidity and ataxia.

Current treatment strategies for PD focus on restoring the depletion of dopamine, generally through the administration of the dopamine precursor L-DOPA (L-3-4-dihydroxyphenylalanine). L-DOPA, (the blood-brain barrier (BBB) penetrating precursor of dopamine), successfully increases the synthesis and release of dopamine. However, as the disease progresses, less dopaminergic neurons are available to synthesize dopamine from the precursor and the effectiveness of the treatment decreases whilst L-DOPA-induced dyskinesia appears. Other treatments with dopamine agonists, monoamine oxidants inhibitor or COMT inhibitors also demonstrate partial improvement but they cannot prevent progression of the disease.

Cell transplantation has been suggested as an alternative treatment option for repairing and replacing missing dopaminergic neurons. For such cell replacement therapy to work, implanted cells must survive and integrate, both functionally and structurally, within the damaged tissue.

The use of stem cells as a cellular source in cell replacement therapy for Parkinson's disease has been recently suggested. Stem cells have the ability to exist in vivo in an undifferentiated state and to self-renew. They are not restricted to cell types specific to the tissue of origin, and so they are able to differentiate in response to local environmental cues from other tissues. This capability of self renewal and differentiation has great therapeutic potential in curing diseases.

In Parkinson's disease the stem cell replacement strategy is based on the idea that restoration of dopamine (DA) neurotransmission is effected by cell grafts that integrate over time into the remaining tissue and produce a long-lasting functional tissue. There are two methods of treating stem cells for grafting in PD. In the first method, prior to transplantation, cells are differentiated in-vitro to dopaminergic neurons. This allows for standardization and quality-control of the relevant cells. The second method comprises transplantation of undifferentiated stem cells that are thought to differentiate in-vivo to dopaminergic neurons following implantation into the striatum or substantia nigra.

In theory, DA neurons for cell therapy in PD could be made from stem cells from four different sources: fetal dopaminergic neurons, neural stem cells, embryonic stem cells and bone marrow stem cells.

Bone marrow contains two major populations of stem cells: hematopoietic stem cells (HSCs) and mesenchymal stem cells (MSCs) occasionally referred to as bone marrow stromal cells.

Rat BMSC following differentiation were shown to express Tyrosine-hydroxylase (TH), choline acetyltransferase and beta-III tubulin [Woodbury, D., et al., J Neurosci Res. 69(6):908-17, 2002]. Clinical therapeutic potential, of mouse BMSC in PD was demonstrated by injecting mouse BMSC intrastriatally to 1-methyl-4-phenyl-1,2,3,6-tetrahydropyridine (MPTP) mouse model of PD. The transplanted cells survived and expressed TH. Moreover improvement on the rotarod test at 35 days following transplantation was indicated [Li, Y., et al., Neurosci Lett. 316(2):67-70, 2001].

U.S. Patent Appl. 20050265983 teaches human dopamine synthesizing MSCs which expressed neuronal markers and transcription factors that characterize midbrain DA neuron following induction of neuronal differentiation.

Differentiation of MSCs in the presence of BDNF and retinoic acid is taught by Hermann A, et al., J Cell Sci 117: 4411-4422 and Sanchez-Ramos et al., Exp Neurol 164:247-256.

### SUMMARY OF THE INVENTION

According to an aspect of some embodiments of the present invention there is provided a method of generating a cell which secretes dopamine, the method comprising incubating mesenchymal stem cells in a culture medium comprising brain derived neurotrophic factor (BDNF), wherein the culture medium comprises no more than 0.1 μM retinoic acid (RA).

According to some embodiments of the invention, the culture medium is devoid of retinoic acid.

According to an aspect of some embodiments of the present invention there is provided a method of generating a cell which secretes dopamine, the method comprising incubating mesenchymal stem cells in a culture medium comprising glial-derived neurotrophic factor GDNF, transforming growth factor β3 (TGFβ3) and retinoic acid (RA).

According to some embodiments of the invention, the culture medium further comprises a component selected from the group consisting of B27, dibutyryl cyclic AMP (dbcAMP), 3-isobutyl-1-methyl-xanthine (IBMX) and ascorbic acid.

According to some embodiments of the invention, the culture medium further comprises B27, dibutyryl cyclic AMP (dbcAMP), 3-isobutyl-1-methyl-xanthine (IBMX) and ascorbic acid.

According to some embodiments of the invention, the method further comprises culturing the mesenchymal stem cells in a pre-differentiation medium, the pre-differentiation medium comprising fibroblast growth factor 2 (FGF-2) and epidermal growth factor (EGF).

According to some embodiments of the invention, the pre-differentiation medium further comprises N2 supplement.

According to some embodiments of the invention, the culture medium is devoid of antioxidants.

According to some embodiments of the invention, the culture medium comprises polyunsaturated fatty acids.

According to some embodiments of the invention, the mesenchymal stem cells are not genetically modified.

According to some embodiments of the invention, the method further comprises isolating dopamine secreting cells following the incubating.

According to an aspect of some embodiments of the present invention there is provided an isolated cell population comprising differentiated mesenchymal stem cells secreting more than 800 pg of dopamine per million cells following KCl depolarization, the cells being non-genetically modified.

According to some embodiments of the invention, at least 30% of the cells express tyrosine hydroxylase (TH).

According to some embodiments of the invention, the differentiated mesenchymal stem cells express higher levels of Tuj 1 than in non-differentiated mesenchymal stem cells.

According to some embodiments of the invention, the differentiated mesenchymal stem cells express neuronal nuclear specific antigen (NeuN).

According to some embodiments of the invention, the differentiated mesenchymal stem cells express Nurr1.

According to some embodiments of the invention, the differentiated mesenchymal stem cells express lower levels of GAD67 than in non-differentiated mesenchymal stem cells.

According to some embodiments of the invention, the mesenchymal stem cells are differentiated in a culture medium comprising brain derived neurotrophic factor (BDNF), wherein the culture medium comprises no more than about 0.01 μM retinoic acid (RA).

According to some embodiments of the invention, the mesenchymal stem cells are differentiated in a culture medium comprising glial-derived neurotrophic factor GDNF, transforming growth factor β3 (TGFβ3) and retinoic acid (RA).

According to an aspect of some embodiments of the present invention there is provided a pharmaceutical composition comprising an isolated cell population comprising differentiated mesenchymal stem cells secreting more than 800 pg of dopamine per million cells following KCl depolarization, the cells being non-genetically modified.

According to an aspect of some embodiments of the present invention there is provided a method of treating a neurodegenerative disorder in a subject in need thereof, comprising transplanting a therapeutically effective amount of the pharmaceutical composition of the present invention into the subject, thereby treating the neurodegenerative disorder.

According to some embodiments of the invention, the neurodegenerative disorder is selected from the group consisting of Parkinson's disease, multiple sclerosis, amyotrophic lateral sclerosis (ALS), autoimmune encephalomyelitis, Alzheimer's disease, Stroke and Huntington's disease.

According to some embodiments of the invention, the neurodegenerative disorder is Parkinson's disease.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D are bar graphs and photographs illustrating tyrosine hydroxylase (TH) expression following various induction protocols. (A) Western blot results of TH expression normalized to β-actin following differentiation protocols that included either BDNF or GDNF and TGFβ3, with or without the addition of RA (means±STD,* p<0.05). (B) Western blot results of TH expression normalized to emerin following differentiation protocols that included either BDNF or GDNF, TGFβ3 and RA (means±STD,* p<0.05). (C-D) Immunocytochemistry of TH and DAPI nuclear stain.

FIGS. 2A-F are photographs illustrating the mesenchymal stem cells (MSCs) prior to and following BDNF-mediated differentiation. (A) Fibroblast-like morphology of MSCs. (B) Neuronal morphology of MSCs following differentiation (C-F). Immunocytochemistry for neuronal markers Tuj1 and NeuN in differentiated cells and untreated MSCs.

FIGS. 3A-D are graphs illustrating the dopaminergic characteristics of differentiated MSCs. (A-B) Real-time PCR analysis of the dopaminergic transcription factor Nurr1 and the GABAergic marker GAD67. (C) Intracellular FACS analysis of TH in untreated and differentiated MSCs (black line represents PE-conjugated isotype control; green and pink lines represent anti-TH staining). (D) HPLC analysis of dopamine secreted by MSCs and differentiated cells to the conditioned medium (growth medium/differentiation medium) and to the KCL HBSS depolarization buffer (means±STD).

### DESCRIPTION OF SPECIFIC EMBODIMENTS OF THE INVENTION

The present invention, in some embodiments thereof, relates to methods of generating dopamine secreting cells and to uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Neurodegenerative disorders which are characterized by loss of neuronal functions, such as Parkinson's disease, cannot be efficiently treated using conventional drug therapy since such drugs have no effect on the underlying disease process which is typically caused by neuronal degeneration. Consequently, drug therapy cannot fully compensate for the increasing loss of neuronal cells.

The specificity of the damaged cells makes Parkinson's disease an attractive candidate for cell transplantation therapy. However, although the embryonic stem cell field has made impressive progress in establishing methods to generate dopaminergic neurons in vitro, and has even had limited success in animal models, teratoma formation remains a great concern. Moreover, the use of fetal or embryonic stem cells, though ideal, not only requires allograft transplantation with its inherent risk of immune rejection, it also poses major ethical concerns.

Bone marrow mesenchymal stem cells (MSCs) are known for their ability to adhere to cell culture plastic surfaces and to expand and proliferate extensively. Under specific conditions, MSCs can differentiate into various mesenchymal phenotypes, such as bone, cartilage, and fat. Several studies have shown that both human and rodent MSCs are also capable of differentiating into neuron-like cells in vitro.

Whilst reducing the present invention to practice, the present inventors have found culturing conditions under which mesenchymal stem cells (MSCs) may be differentiated into cells having a neuronal phenotype and capable of secreting large amounts of dopamine. Accordingly, the present inventors propose that such differentiated MSCs can be used to treat patients with neurodegenerative diseases following transplantation.

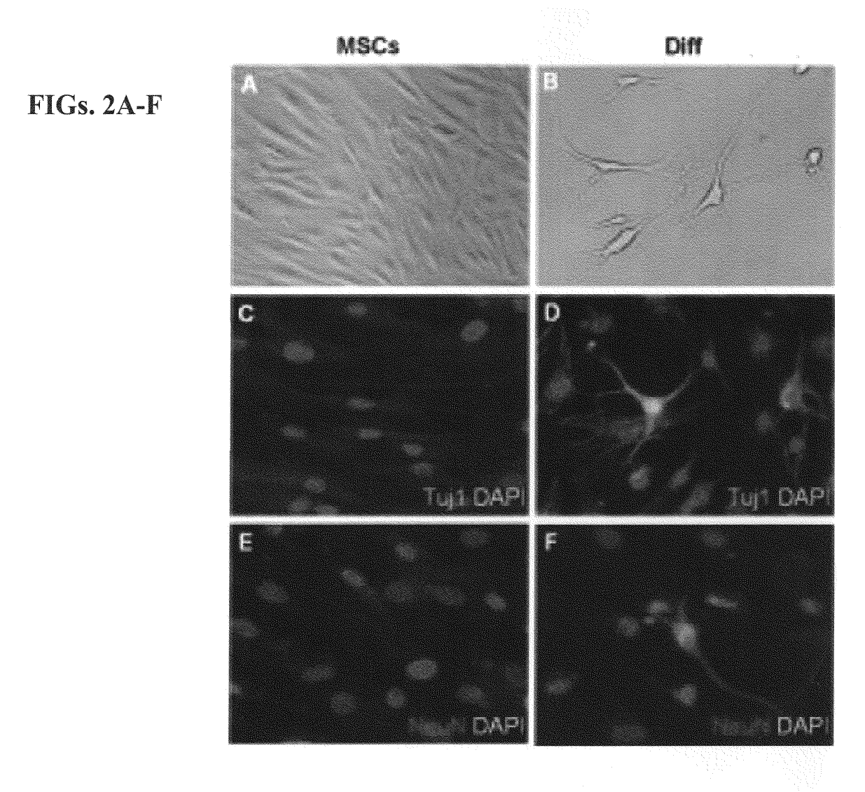

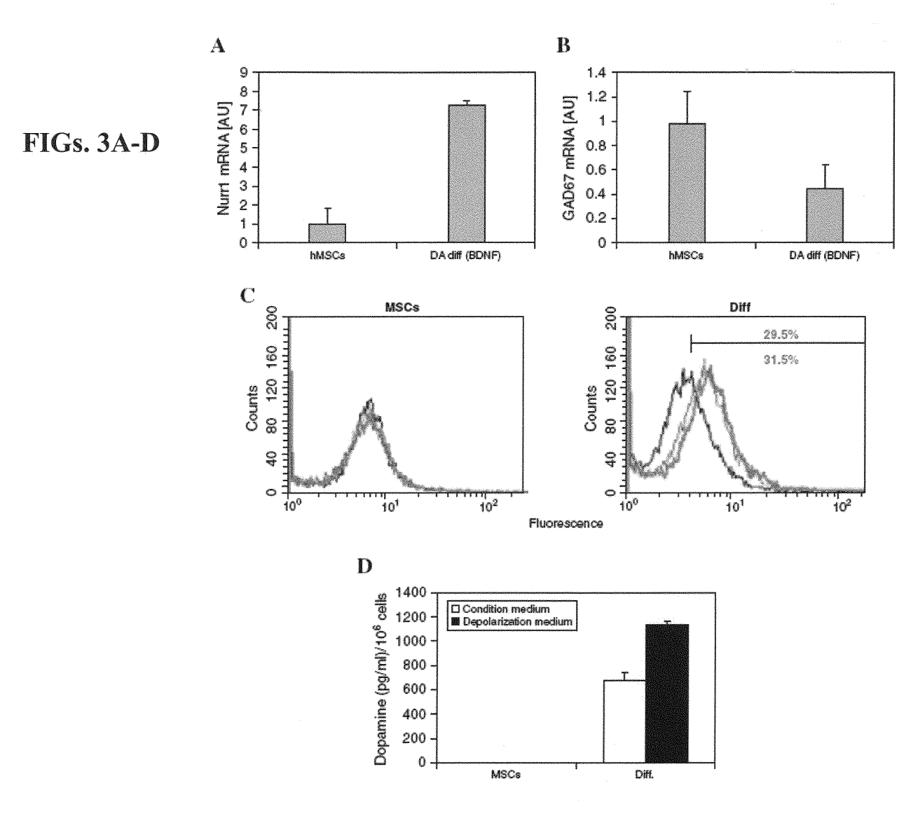

The present inventors have shown that human MSCs differentiated according to particular protocols represent a neuronal-like morphology (FIG. 2B) accompanied by the ability to secrete dopamine (FIG. 3D).

Thus, according to one aspect of the present invention there is provided a method of generating a cell which secretes dopamine, the method comprising incubating mesenchymal stem cells in a culture medium comprising brain derived neurotrophic factor (BDNF), wherein the culture medium comprises no more than 0.1 μM retinoic acid (RA) or in a culture medium comprising glial-derived neurotrophic factor GDNF, transforming growth factor β3 (TGFβ3) and retinoic acid (RA).

The term “mesenchymal stem cell” or “MSC” refers to fetal or postnatal (e.g., adult) cells which irreversibly differentiate (either terminally or non-terminally) to give rise to cells of a mesenchymal cell lineage and which are also capable of dividing to yield stem cells. The mesenchymal stem cells of the present invention may be of a syngeneic or allogeneic source, although the first is preferred.

According to one embodiment of this aspect of the present invention the mesenchymal stem cells are not genetically modified (i.e. transformed with an expression construct) to generate the cells and cell populations described herein.

According to another embodiment of this aspect of the present invention, the mesenchymal stem cells are genetically modified—e.g. with a construct for expression of tyrosine hydroxylase.

It will be appreciated that the cells of the present invention may be derived from any stem cell, although preferably not ES cells.

Mesenchymal stem cells may be isolated from various tissues including but not limited to bone marrow, peripheral blood, blood, placenta and adipose tissue. A method of isolating mesenchymal stem cells from peripheral blood is described by Kassis et al [Bone Marrow Transplant. 2006 May; 37(10):967-76]. A method of isolating mesenchymal stem cells from placental tissue is described by Zhang et al [Chinese Medical Journal, 2004, 117 (6):882-887]. Methods of isolating and culturing adipose tissue, placental and cord blood mesenchymal stem cells are described by Kern et al [Stem Cells, 2006; 24:1294-1301].

According to a preferred embodiment of this aspect of the present invention, the mesenchymal stem cells are human.

Bone marrow can be isolated from the iliac crest of an individual by aspiration. Low-density BM mononuclear cells (BMMNC) may be separated by a FICOL-PAGUE density gradient. In order to obtain mesenchymal stem cells, a cell population comprising the mesenchymal stem cells (e.g. BMMNC) may be cultured in a proliferating medium capable of maintaining and/or expanding the cells. According to one embodiment the populations are plated on polystyrene plastic surfaces (e.g. in a flask) and mesenchymal stem cells are isolated by removing non-adherent cells. Alternatively mesenchymal stem cell may be isolated by FACS using mesenchymal stem cell markers.

Preferably the MSCs are at least 50% purified, more preferably at least 75% purified and even more preferably at least 90% purified.

Following isolation the cells are typically expanded by culturing in a proliferation medium capable of maintaining and/or expanding the isolated cells ex vivo as described in Example 1 hereinbelow. The proliferation medium may be DMEM, alpha-MEM or DMEM/F12. Preferably, the proliferation medium is DMEM. Preferably, the proliferation medium further comprises SPN, L-glutamine and a serum (such as fetal calf serum or horse serum) such as described in Example 1, herein below.

Differentiation to dopamine-secreting cells can be effected by incubating the MSCs in culture medium comprising:

(a) BDNF; or

(b) GDNF, TGFβ3 and RA

The differentiating media may be DMEM or DMEM/F12, or any other medium that supports neuronal growth. According to a preferred embodiment of this aspect of the present invention, the medium is Neurobasal medium (e.g. Cat. No. 21103049, Invitrogen, Ca, U.S.A.).

Preferably, the MSCs are differentiated for a period of time between about 5 days to about 13 days in the differentiating medium so that differentiation into dopamine-secreting cells may occur. The exact number of days is dependent upon the particular differentiating agents added to the medium and may be determined empirically.

According to one embodiment of this aspect of the present invention, the cells are incubated (e.g. for about 7 days) in a culture medium comprising BDNF (e.g. 50 ng/ml).

BDNF is commercially available from such Companies as R&D Systems, Cell Sciences and Promokine.

It will be appreciated that the culture medium may comprise other components such as supplements (e.g. B27); other differentiating agents (e.g. dibutyryl cyclic AMP (dbcAMP) and ascorbic acid; and antibiotics, (e.g. 3-isobutyl-1-methyl-xanthine (IBMX)) all of which are available from all from Sigma, St Louis, Mo. According to this aspect of the present invention, the culture medium comprises less than about 0.5 μM retinoic acid (RA). Preferably, the cells comprise less than about 0.1 μM RA, less than 0.05 μM RA. According to one embodiment, the culture medium is devoid of RA completely.

According to another embodiment of this aspect of the present invention, the cells are incubated (e.g. for about 7 days) in a culture medium comprising GDNF (e.g. 10 ng/ml), TGFβ3 and RA (e.g. 0.1 μM).

GDNF is commercially available from such Companies as R&D Systems, Cell Sciences and Promokine.

TGFβ3 is commercially available from such Companies as R&D Systems.

All-trans retinoic acid is commercially available from companies such as Sigma, St Louis, Mo.

It will be appreciated that the culture medium which comprises the three named differentiating agents may also comprise other components such as supplements (e.g. B27); other differentiating agents (e.g. dibutyryl cyclic AMP (dbcAMP) and ascorbic acid; and antibiotics, (e.g. 3-isobutyl-1-methyl-xanthine (IBMX)) all of which are available from Sigma, St Louis, Mo.

According to one embodiment differentiation in the culture mediums described herein above is effected in the presence of at least one type of long-chain polyunsaturated fatty acids (PUFA). Long-chain polyunsaturated fatty acids, such as docosahexaenoic acid (DHA) and arachidonic acid (AA), are known to be essential for proper neuronal development and function. DHA has been shown to modulate the biosynthesis of phosphatidyl serine (PS) one of the major anionic phospholipids in neuronal membranes [Green and Yavin, J. Neurochem. 65: 2555-2560, 1995; Garcia et al., J. Neurochem. 70:24-30, 1988]. In neuronal cell culture studies it has been demonstrated that DHA has antiapoptotic effects, probably related to DHA-induced PS accumulation (Kim et al., J. Biol. Chem. 275: 35215-35223, 2000; Kim et al., J. Mol. Neurosci. 16: 223-227, 2002; and Akbar and Kim, J. Neurochem. 82: 655-665, 2002).

Mesenchymal stem cells may be incubated in a “pre-differentiation medium” for at least 2 days, preferably 3 days, prior to their incubation in the differentiation mediums of the present invention in order to predispose the cells to differentiate into dopamine-secreting cells.

The “pre-differentiation medium” according to this aspect of the present invention may comprises differentiating agents such as N₂ supplement, commercially available from Invitrogen, New Haven, Conn., fibroblast growth factor 2 (e.g. 20 ng/ml) and epidermal growth factor (e.g. 20 ng/ml), the latter two being commercially available from R&D Systems, Minneapolis, Minn.).

As used herein, “N2 supplement” refers to a mixture of components comprising about 5 μg/ml insulin; 20 n-M progesterone; 100 μM putrescine; 30 nM selenium; and 100 μg/ml transferrin. N2 supplement is wildely available from such Companies as e.g. Sigma Aldrich and Invitrogen, Carlsbad, Calif.

According to one embodiment of this aspect of the present invention, the culture medium is devoid of antioxidants, such as for example butylated hydroxyanisole (BHA) and N-acetylcysteine (NAC) and other chemical agents such as DMSO.

Once differentiated, the cells can be tested for their ability to secrete dopamine. A high performance liquid chromatography (HPLC) procedure for analyzing the amount of dopamine secreted by cells is described in the Examples section below.

Following differentiation and optional testing, the cell populations may be enriched for cells with a particular phenotype (e.g. expression of tyrosine hydroxylase (TH), expression of NeuN, expression of Nurr1 and/or expression of GAD67. This may be effected by FACS using an antibody against one of the above-mentioned markers. Antibodies capable of recognizing these markers are available for example from Sigma, Millipore and Abcam. If the cell marker is an internal marker, preferably the FACS analysis comprises antibodies or fragments thereof which may easily penetrate a cell and may easily be washed out of the cell following detection. The FACS process may be repeated a number of times using the same or different markers depending on the degree of enrichment and the cell phenotype required as the end product.

Non-genetically modified mesesnchymal stem cells differentiated according to the above described protocols typically secrete more than 800 pg of dopamine per million cells following KCl depolarization.

Thus, according to another aspect of the present invention there is provided an isolated cell population comprising differentiated mesenchymal stem cells secreting more than 800 pg of dopamine per million cells following KCl depolarization, the cells being non-genetically modified.

The term “isolated” as used herein refers to a population of cells that has been removed from its in-vivo location (e.g. bone marrow, neural tissue). Preferably the isolated cell population is substantially free from other substances (e.g., other cells) that are present in its in-vivo location.

KCl depolarization is effected by incubating the cell population of the present invention in a depolarization buffer containing 56 mM KCl in Hanks' balanced salt solution (HBSS; Biological Industries) for 30 minutes.

According to one embodiment, the mesenchymal stem cells secrete more than 850 pg of dopamine per million cells following KCl depolarization. According to another embodiment, the mesenchymal stem cells secrete more than 900 pg of dopamine per million cells following KCl depolarization. According to one embodiment, the mesenchymal stem cells secrete more than 950 pg of dopamine per million cells following KCl depolarization. According to one embodiment, the mesenchymal stem cells secrete about 1 ng of dopamine per million cells following KCl depolarization.

Cell populations obtained according to the two protocols described herein are typically non-homogeneous. However, typically at least 30% of the cells express tyrosine hydroxylase (TH)—NP_{—}000351.

According to one embodiment, the differentiated mesenchymal stem cells express higher levels (e.g. 10% higher, 20% higher or even 50% higher) of Neuron specific beta III Tubulin (Tuj1; Swiss Prot. No. Q13509) than in non-differentiated mesenchymal stem cells.

According to another embodiment, the differentiated mesenchymal stem cells express neuronal nuclear specific antigen (NeuN; Development. 1992 September; 116(1):201-11).

According to still another embodiment, the differentiated mesenchymal stem cells express Nurr1 (Swiss Prot. No. P43354).

According to still another embodiment, the differentiated mesenchymal stem cells express lower levels (e.g. 10% lower, 20% lower or even 50% lower) of GAD67 (Swiss Prot. No. Q99259) than in non-differentiated mesenchymal stem cells.

Methods of ascertaining if the cells express particular markers or levels of markers are well known in the art including for example, HPLC, Western Blot analysis, immunohistochemistry, RT-PCR, Real-time RT-PCT and Northern Blot Analysis.

It will be appreciated that the percentage of cells which express a particular marker may be raised or lowered according to the intended needs using techniques such as flow cytometry

The cells and cell populations of the present invention may be useful for a variety of therapeutic purposes, such as treatment of neurodegenerative disorders.

As used herein, the phrase “neurodegenerative disorder” refers to any disorder, disease or condition of the nervous system (preferably CNS) which is characterized by gradual and progressive loss of neural tissue, neurotransmitter, or neural functions. Examples of neurodegenerative disorder include, Parkinson's disease, multiple sclerosis, amyotrophic lateral sclerosis (ALS), autoimmune encephalomyelitis, Alzheimer's disease, stroke and Huntington's disease.

In any of the methods described herein the cells may be obtained from any autologous or non-autologous (i.e., allogeneic or xenogeneic) human donor. For example, cells may be isolated from a human cadaver or a donor subject.

The cells of the present invention can be administered to the treated individual using a variety of transplantation approaches, the nature of which depends on the site of implantation.

The term or phrase “transplantation”, “cell replacement” or “grafting” are used interchangeably herein and refer to the introduction of the cells of the present invention to target tissue. The cells can be derived from the recipient or from an allogeneic or xenogeneic donor.

The cells can be grafted into the central nervous system or into the ventricular cavities or subdurally onto the surface of a host brain. Conditions for successful transplantation include: (i) viability of the implant; (ii) retention of the graft at the site of transplantation; and (iii) minimum amount of pathological reaction at the site of transplantation. Methods for transplanting various nerve tissues, for example embryonic brain tissue, into host brains have been described in: “Neural grafting in the mammalian CNS”, Bjorklund and Stenevi, eds. (1985); Freed et al., 2001; Olanow et al., 2003). These procedures include intraparenchymal transplantation, i.e. within the host brain (as compared to outside the brain or extraparenchymal transplantation) achieved by injection or deposition of tissue within the host brain so as to be opposed to the brain parenchyma at the time of transplantation.

Intraparenchymal transplantation can be effected using two approaches: (i) injection of cells into the host brain parenchyma or (ii) preparing a cavity by surgical means to expose the host brain parenchyma and then depositing the graft into the cavity. Both methods provide parenchymal deposition between the graft and host brain tissue at the time of grafting, and both facilitate anatomical integration between the graft and host brain tissue. This is of importance if it is required that the graft becomes an integral part of the host brain and survives for the life of the host.

Alternatively, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura. Injections into selected regions of the host brain may be made by drilling a hole and piercing the dura to permit the needle of a microsyringe to be inserted. The microsyringe is preferably mounted in a stereotaxic frame and three dimensional stereotaxic coordinates are selected for placing the needle into the desired location of the brain or spinal cord. The cells may also be introduced into the putamen, nucleus basalis, hippocampus cortex, striatum, substantia nigra or caudate regions of the brain, as well as the spinal cord.

The cells may also be transplanted to a healthy region of the tissue. In some cases the exact location of the damaged tissue area may be unknown and the cells may be inadvertently transplanted to a healthy region. In other cases, it may be preferable to administer the cells to a healthy region, thereby avoiding any further damage to that region. Whatever the case, following transplantation, the cells preferably migrate to the damaged area.

For transplanting, the cell suspension is drawn up into the syringe and administered to anesthetized transplantation recipients. Multiple injections may be made using this procedure.

The cellular suspension procedure thus permits grafting of the cells to any predetermined site in the brain or spinal cord, is relatively non-traumatic, allows multiple grafting simultaneously in several different sites or the same site using the same cell suspension, and permits mixtures of cells from different anatomical regions. Multiple grafts may consist of a mixture of cell types, and/or a mixture of transgenes inserted into the cells. Preferably from approximately 10⁴ to approximately 10⁸ cells are introduced per graft.

For transplantation into cavities, which may be preferred for spinal cord grafting, tissue is removed from regions close to the external surface of the central nerve system (CNS) to form a transplantation cavity, for example as described by Stenevi et al. (Brain Res. 114:1-20., 1976), by removing bone overlying the brain and stopping bleeding with a material such a gelfoam. Suction may be used to create the cavity. The graft is then placed in the cavity. More than one transplant may be placed in the same cavity using injection of cells or solid tissue implants. Preferably, the site of implantation is dictated by the CNS disorder being treated.

Since non-autologous cells are likely to induce an immune reaction when administered to the body several approaches have been developed to reduce the likelihood of rejection of non-autologous cells. Furthermore, since diseases such as multiple sclerosis are inflammatory based diseases, the problem of immune reaction is exacerbated. These include either suppressing the recipient's immune system, providing anti-inflammatory treatment and/or encapsulating the non-autologous cells in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol. Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Technol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE™), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

In any of the methods described herein, the cells can be administered either per se or, preferably as a part of a pharmaceutical composition that further comprises a pharmaceutically acceptable carrier.

As used herein a “pharmaceutical composition” refers to a preparation of one or more of the chemical conjugates described herein, with other chemical components such as pharmaceutically suitable carriers and excipients. The purpose of a pharmaceutical composition is to facilitate administration of a compound to a subject.

Hereinafter, the term “pharmaceutically acceptable carrier” refers to a carrier or a diluent that does not cause significant irritation to a subject and does not abrogate the biological activity and properties of the administered compound. Examples, without limitations, of carriers are propylene glycol, saline, emulsions and mixtures of organic solvents with water.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

According to a preferred embodiment of the present invention, the pharmaceutical carrier is an aqueous solution of saline.

Techniques for formulation and administration of drugs may be found in “Remington's Pharmaceutical Sciences,” Mack Publishing Co., Easton, Pa., latest edition, which is incorporated herein by reference.

Suitable routes of administration include direct administration into the tissue or organ of interest. Thus, for example the cells may be administered directly into the brain as described herein above.

For any preparation used in the methods of the invention, the therapeutically effective amount or dose can be estimated initially from in vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. For example, 6-OHDA-lesioned mice may be used as animal models of Parkinson's. In addition, a sunflower test may be used to test improvement in delicate motor function by challenging the animals to open sunflowers seeds during a particular time period.

Transgenic mice may be used as a model for Huntingdon's disease which comprise increased numbers of CAG repeats have intranuclear inclusions of huntingtin and ubiquitin in neurons of the striatum and cerebral cortex but not in the brain stem, thalamus, or spinal cord, matching closely the sites of neuronal cell loss in the disease.

Transgenic mice may be used as a model for ALS disease which comprise SOD-1 mutations.

The septohippocampal pathway, transected unilaterally by cutting the fimbria, mimics the cholinergic deficit of the septohippocampal pathway loss in Alzheimers disease. Accordingly animal models comprising this lesion may be used to test the cells of the present invention for treating Alzheimers.

Animal models of demyelinating diseases include shiverer (shi/shi, MBP deleted) mouse, MD rats (PLP deficiency), Jimpy mouse (PLP mutation), dog shaking pup (PLP mutation), twitcher mouse (galactosylceramidase defect, as in human Krabbe disease), trembler mouse (PMP-22 deficiency). Virus induced demyelination model comprise use if Theiler's virus and mouse hepatitis virus. Autoimmune EAE is a possible model for multiple sclerosis.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1). For example, a multiple sclerosis patient can be monitored symptomatically for improved motor functions indicating positive response to treatment.

For injection, the active ingredients of the pharmaceutical composition may be formulated in aqueous solutions, preferably in physiologically compatible buffers such as Hank's solution, Ringer's solution, or physiological salt buffer.

Dosage amount and interval may be adjusted individually to levels of the active ingredient which are sufficient to effectively regulate the neurotransmitter synthesis by the implanted cells. Dosages necessary to achieve the desired effect will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition. For example, a treated multiple sclerosis patient will be administered with an amount of cells which is sufficient to alleviate the symptoms of the disease, based on the monitoring indications.

The cells of the present invention may be co-administered with therapeutic agents useful in treating neurodegenerative disorders, such as gangliosides; antibiotics, neurotransmitters, neurohormones, toxins, neurite promoting molecules; and antimetabolites and precursors of neurotransmitter molecules such as L-DOPA. Additionally, the cells of the present invention may be co-administered with other cells capable of synthesizing a neurotransmitter and/or secreting a neurotrophic factor. Such cells are known in the art including, but not limited to those described in U.S. Pat. App. No. 20050265983 and U.S. Pat. App. No. 20090010895.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non limiting fashion.

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

MSC isolation, culture, and characterization: Fresh bone marrow aspirates harvested from the iliac crests of adult donors (age 19-76) were diluted with Hanks' balanced salt solution. Mononuclear cells were isolated by centrifugation in Unisep-Maxi tubes (Novamed, Jerusalem, Israel) by density gradient. The cells were then plated in Dulbecco's Modified Eagle's Medium (DMEM) supplemented with 15% fetal bovine serum (FBS), 2 mM glutamine, 100 μg/ml streptomycin, 100 units/ml penicillin, 12.5 units/ml nystatin (Biological Industries, Beit HaEmek, Israel) in polystyrene plastic 75-cm² tissue-culture flasks. After 24 hours, nonadherent cells were removed. The medium was changed every 3-4 days. Adherent cells were cultured to 70%-90% confluence and reseeded at a density of 5,000-10,000 cells/cm². The cells were maintained at 37° C. in a humidified 5% CO₂ incubator. The MSCs were characterized for their cell-surface phenotype and their mesenchymal differentiation capacity, as previously described (Blondheim er al., 2006, Stem Cells Dev 15:141-164).

Induction of differentiation: The differentiation protocol was composed of 2 steps. In step 1, cells were transferred to serum-free medium (DMEM) supplemented with 2 mM glutamine, 100 μg/ml streptomycin, 100 units/ml penicillin, 12.5 units/ml nystatin (Biological Industries), N₂ supplement (Invitrogen, New Haven, Conn.) and 20 ng/ml fibroblast growth factor 2 and epidermal growth factor (both from R&D Systems, Minneapolis, Minn.) for 48-72 hours. In step 2, the medium was changed to Basic induction medium containing Neurobasal, B27 (both from Invitrogen), in addition to 1 mM dibutyryl cyclic AMP (dbcAMP), 3-isobutyl-1-methyl-xanthine (IBMX), and 200 μM ascorbic acid (all from Sigma, St Louis, Mo.). The following factors were added to the Basic induction medium in different combinations: brain-derived neurotrophic factor (BDNF; 50 ng/ml), glial-derived neurotrophic factor (GDNF;10 ng/ml), neurturin (100 ng/ml), neurotrophin 3 (NT3; 50 ng/ml), and fibroblast growth factor 8 (FGF8; 100 ng/ml (all from Cytolab, Rehovot, Israel); transforming growth factor β3 (TGFβ3; 2 ng/ml), sonic hedgehog (Shh; 500 ng/ml, both from R&D); estrogen (0.1 μM, Sigma); and all-trans-retinoic acid (RA; 0.1 μM, Sigma).

Western blot: Total protein was extracted by suspending the harvested cells in lysis buffer containing 10 mM Tris base (U.S. Biochemical Corporation, Cleveland, Ohio), 5 mM EDTA (Merck, Whitehouse Station, N.J.), 140 mM sodium chloride (NaCl; BioLab, Jerusalem, Israel), 10 mM sodium fluoride (NaF; Sigma), 0.5% NP 40 (U.S. Biochemical Corporation), and 1 μM phenylmethylsulfonyl fluoride (PMSF; Sigma). Following incubation on ice for 30 minutes, the mixture was centrifuged and the supernatants were collected. Protein content was determined by the BCA protein assay kit (Pierce, Rockford, Ill.). Twenty-five micrograms of protein from each sample were subjected to sodium dodecyl-sulfate (SDS)-polyacrylamide gel electrophoresis (PAGE) (12.5% acrylamide), followed by electrophoretic transfer to nitrocellulose membrane (Schleicher & Schuell, Dassel, Germany). Membranes were probed by mouse anti-TH (1:10,000, Sigma) and mouse anti β-actin (1:10,000, Chemicon, Billerica, Mass.) and then by goat anti-mouse horseradish peroxidase conjugated antibody (1:10,000) (Jackson, West Grove, Pa.) and rabbit anti-emerin (1:5,000, Santa Cruz Biotechnology, Santa Cruz, Calif.), followed by goat anti-rabbit horseradish peroxidase conjugated secondary antibody (1:10,000, Sigma). Adequate controls included HeLa cells (ATCC, Manassas, Va.) as negative control and neuroblastoma M17 cells (BE (2)-M17, ATCC) as positive control. Proteins of interest were detected using the enhanced Super Signal® chemiluminescent detection kit (Pierce).

Immunocytochemistry: Cells were fixed with 4% paraformaldehyde, blocked, and permeabelized in 5% goat serum (Biological Industries), 1% bovine serum albumin (Sigma), and 0.5% Triton-X in PBS for 1 hour at room temperature. Primary antibodies included mouse anti-TH and mouse anti-β3-tubulin (both 1:1000, Sigma), followed by goat anti-mouse Alexa-488 or Alexa-568 (both 1:1000, Molecular Probes). DNA-specific fluorescent dye 4,6-diamidino-2-phenylindole (DAPI; Sigma) counterstain was used to detect cell nuclei. Cells were photographed with a fluorescence Olympus IX70-S8F2 microscope with a fluorescent light source (excitation wavelength, 330-385 nm; barrier filter, 420 nm) and a U-MNU filter cube (Olympus, Center Valley, Pa.).

RNA isolation and cDNA synthesis: Total RNA was isolated by a commercial TriReagent (Sigma). The amount and quality of RNA was determined with the ND-1000 spectrophotometer (NanoDrop, Wilmington, Del.). First-strand cDNA synthesis was carried out with Super Script II RNase H-reverse transcriptase (Invitrogen) using a random primer.

Real-time quantitative reverse transcription polymerase chain reaction (PCR): Real-time quantitative PCR of the desired genes was performed in an ABI Prism 7700 sequence detection system (Applied Biosystems, Foster City, Calif.) using Syber Green PCR Master Mix (Applied Biosystems) and the following primers: glyceraldehyde-3-phosphate dehydrogenase (GAPDH) sense CGACAGTCAGCCGCATCTT (SEQ ID NO: 1), GAPDH antisense CCAATACGACCAAATCCGTTG (SEQ ID NO: 2); Nurr1 sense GGATGGTCAAAGAAGTGGTTCG (SEQ ID NO: 3), Nurr1 antisense CCTGTGGGCTCTTCGGTTT (SEQ ID NO: 4); glutamic acid decarboxylase 67 (GAD67) sense CGAGGACTCTGGACAGTAGAGG (SEQ ID NO: 5), GAD67 antisense GATCTTGAGCCCCAGTTTTCTG (SEQ ID NO: 6); The GAPDH gene served as an internal control. For each gene, the specificity of the PCR product was assessed by verifying a single peak in the melting curve analysis. The PCR was performed in a total volume of 20 μl containing 1 μl of the previously described cDNA, the 3′ and 5′ primers at a final concentration of 500 nM each, and 10 μl of Syber Green Mix. The amplification protocol was 40 cycles of 95° C. for 15 seconds followed by 60° C. for 1 minute each. Quantitative calculations of the gene of interest versus GAPDH were done according to the ΔΔCT method, as instructed in the user bulletin of the ABI Prism 7700 sequence detection system (updated 10/2001).

Intracellular fluorescence-activated cell sorter (FACS) analysis: Cells were harvested from the tissue culture flasks, centrifuged, and resuspended in phosphate-buffered saline (PBS). Intracellular staining was performed with the IntraCyte kit (Orion Biosolutions, Vista, Calif.), according to the manufacturer's instructions. TH staining was performed with mouse anti-TH antibody (1:1000, Sigma) followed by donkey anti-mouse phycoerythrin (PE)-conjugated IgG (Jackson Laboratories, Bar Harbor, Me.). The results were analyzed with CellQuest software. A PE-conjugated isotype control was included in each experiment. To verify specific detection of TH expression, we employed HeLa cells as negative control and PC12 (ATCC) cells as positive control.

High performance liquid chromotography (HPLC): Reverse-phase HPLC coupled with an electrochemical detector (ECD) was used to measure dopamine levels. Briefly, the conditioned media of the MSCs (growth media) and the differentiated neuron-like cells (differentiation media) were harvested, and the cells were incubated in KCL depolarization buffer containing 56 mM KCL in Hanks' balanced salt solution (HBSS; Biological Industries) for 30 minutes. Following collection, all samples were stabilized with 0.1 M perchloric acid/metabisulfite (2 mg/ml) (Sigma) and extracted by aluminum adsorption (Bioanalytical Systems, West Lafayette, Ind.). An aliquot of filtrate was applied to the HPLC/ECD (Bioanalytical Systems) equipped with a catecholamine C18 column (125 mm×4.6 mm) (Hichrom, Berkshire, UK), with the electrode potential set at +0.65 V relative to the Ag/AgCl reference electrode. The mobile phase consisted of a monochloroacetate buffer (150 mM, pH 3) containing 10% methanol, 30 mg/l sodium 1-octanesulfonate, and 2 mM EDTA. The flow rate was 1.2 ml/min. Dopamine was identified by retention time and validated by co-elution with catecholamine standards under varying buffer conditions and detector settings.

Statistics: Statistical significance was determined by one-way ANOVA followed by Dunnett post hoc multiple comparisons.

Results

Phenotypic characterization of human MSCs

The MSCs were characterized according to the previously published position paper by Dominici et al. (Cytotherapy 8:315-317, 2006), expressed mesenchymal markers such as CD29+, CD44+, and CD105+, and did not express the hematopoietic markers CD45− and CD34−. Moreover, the cells were sufficiently induced to differentiate into adipocytes and osteoblasts (data not shown).

Various differentiation protocols upregulate TH expression

Western blot analysis of TH protein expression, normalized to house-keeping gene Actin, revealed that the Basic induction medium was associated with a moderate increase in TH protein level; the addition of neurotrophic factors further facilitated this effect. In particular, supplementation of the Basic induction medium with Shh, TGFβ3, GDNF, BDNF, and estrogen directly affected the TH protein expression level. However, adding more than one differentiation-inducing factor did not necessarily facilitate TH upregulation. The results of 12 different differentiation protocols are summarized in Table 1, herein below.

TABLE 1

| Differentiation agents | TH/actin ratio |
|---|---|
| Basic induction medium | + |
| +SHH | ++++ |
| +SHH + FGF8 | +++ |
| +TGFB3 | +++++ |
| +GDNF | ++++ |
| +GDNF and TGFB3 | ++++++ |
| +BDNF | +++++ |
| +Neurturin + NT3 | ++++ |
| +Neurturin + NT3 + TGFB3 + GDNF + | +++++ |
| BDNF | |
| +Neurturin + NT3 + TGFB3 + GDNF + | ++++ |
| BDNF + Shh + FGF8 | |
| +estrogen | ++++ |
| +GDNF + TGFB3 + BDNF + estrogen | ++++ |

The supplementation of antioxidants such as butylated hydroxyanisole (BHA) and N-acetylcysteine (NAC) did not facilitate upregulation, but rather led to higher levels of cell death in the culture (data not shown).

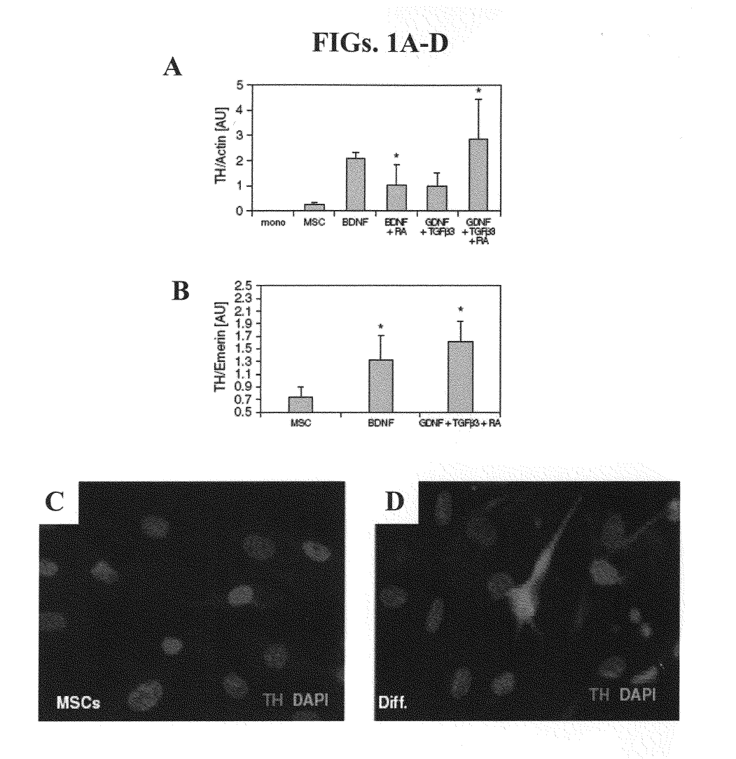

Of the 12 differentiation protocols, the 2 that yielded the highest TH expression were selected: one protocol involved incubation in GDNF, TGFβ3 and RA, and the other, involved incubation in BDNF and RA. Both protocols were carried out in the presence and absence of RA. Western blot analysis of TH protein expression normalized to actin revealed that MSCs expressed basal levels of TH and elevated the TH expression levels following four induction protocols. Although all four induction protocols upregulated TH expression, significant results were obtained with BDNF alone and with the combination of GDNF, TGFβ3, and RA (FIG. 1A). In no experiment was TH expression observed in the starting bone marrow mononuclear cell sample. Conversely, in most experiments, untreated MSCs expressed basal levels of TH.

The TH expression results were verified by normalizing to the nuclear envelope protein emerin as a standard (FIG. 1B). It was found that at 7 days, both differentiation protocols yielded a significant increase in TH expression compared to untreated MSCs. Elongation of the induction process that included incubation in medium containing Shh and FGF8 for 72 hours did not lead to a further increase in TH expression (data not shown). Immunocytochemistry for TH expression confirmed the Western blot analysis (FIGS. 1C,D).

Induction Results in Neuronal Phenotype

The present inventors next sought to examine whether the induction of TH upregulation is correlated with neuronal differentiation of the MSCs. Bright-light microscopy revealed that the use of an induction medium containing BDNF was associated with a morphological change in the cells, from the characteristic MSC fibroblast shape to a neuron-like appearance (FIGS. 2A, B). Immunocytochemistry revealed that the induced cells expressed higher levels of Tuj 1, a neuronal progenitor marker, than the untreated MSCs (FIGS. 2C, D). In addition, the induced cells stained positive for the mature neuronal nuclear specific antigen NeuN (FIGS. 2E, F). No NeuN-positive cells were found following induction with GDNF, TGFβ3, and RA (data not shown).

BDNF-mediated cell differentiation is dopaminergic-specific

The characteristics of the MSCs following incubation with the BDNF-containing differentiation medium were further investigated. Quantitative real time PCR revealed a significant upregulation of the expression of Nurr1, a transcription factor involved in dopaminergic neuron differentiation and maintenance, compared to untreated MSCs (FIG. 3A). Further study indicated down regulation of GAD67, a GABAergic marker, indicating that the induction was dopaminergic-specific (FIG. 3B).

To quantify the yield of the dopamine-directed cells, intracellular FACS analysis was performed of TH expression. More than 30% of the induced cells expressed the TH protein at a detectable level compared to none of the untreated cells (FIG. 3C). HPLC analysis of dopamine secretion showed that prior to induction, dopamine was not detectable in the MSC medium, with or without KCL depolarization. Following induction of differentiation, the cells secreted a mean of 673.69 pg/ml of dopamine per 10⁶ cells to the conditioned media and 1127.86 pg/ml of dopamine per 10⁶ cells to the depolarization buffer (FIG. 3D). For comparison, following induction with GDNF, TGFB and retinoic acid, the cells secreted a mean of 533.5 pg/ml of dopamine per 10⁶ cells to the conditioned media and 997 pg/ml of dopamine per 10⁶ cells to the depolarization buffer.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of generating a cell which secretes dopamine, the method comprising incubating mesenchymal stem cells in a culture medium comprising brain derived neurotrophic factor (BDNF), wherein said culture medium comprises no more than 0.1 μM retinoic acid (RA).

2. The method of claim 1, wherein said culture medium is devoid of retinoic acid.

3. The method of claim 1, wherein said culture medium further comprises a component selected from the group consisting of B27, dibutyryl cyclic AMP (dbcAMP), 3-isobutyl-1-methyl-xanthine (IBMX) and ascorbic acid.

4. The method of claim 1, wherein said culture medium further comprises B27, dibutyryl cyclic AMP (dbcAMP), 3-isobutyl-1-methyl-xanthine (IBMX) and ascorbic acid.

5. The method of claim 1, further comprising culturing said mesenchymal stem cells in a pre-differentiation medium, said pre-differentiation medium comprising fibroblast growth factor 2 (FGF-2) and epidermal growth factor (EGF).

6. The method of claim 5, wherein said pre-differentiation medium further comprises N2 supplement.

7. The method of claim 1, wherein said culture medium is devoid of antioxidants.

8. The method of claim 1, wherein said culture medium comprises polyunsaturated fatty acids.

9. The method of claim 1, wherein said mesenchymal stem cells are not genetically modified.

10. The method of claim 1, further comprising isolating dopamine secreting cells following said incubating.

11. A method of generating a cell which secretes dopamine, the method comprising incubating mesenchymal stem cells in a culture medium comprising glial-derived neurotrophic factor GDNF, transforming growth factor β3 (TGFβ3) and retinoic acid (RA).

12. The method of claim 11, wherein said culture medium further comprises a component selected from the group consisting of B27, dibutyryl cyclic AMP (dbcAMP), 3-isobutyl-1-methyl-xanthine (IBMX) and ascorbic acid.

13. The method of claim 11, wherein said culture medium further comprises B27, dibutyryl cyclic AMP (dbcAMP), 3-isobutyl-1-methyl-xanthine (IBMX) and ascorbic acid.

14. The method of claim 11, further comprising culturing said mesenchymal stem cells in a pre-differentiation medium, said pre-differentiation medium comprising fibroblast growth factor 2 (FGF-2) and epidermal growth factor.

15. The method of claim 14, wherein said pre-differentiation medium further comprises N2 supplement.

16. The method of claim 11, wherein said culture medium is devoid of antioxidants.

17. The method of claim 11, wherein said culture medium comprises polyunsaturated fatty acids.

18. The method of claim 11, wherein said mesenchymal stem cells are non-genetically modified.

19. The method of claim 11, further comprising isolating dopamine secreting cells following said incubating.

20. An isolated cell population comprising differentiated mesenchymal stem cells secreting more than 800 pg of dopamine per million cells following KCl depolarization, the cells being non-genetically modified.

21. The isolated cell population of claim 20, wherein at least 30% of the cells express tyrosine hydroxylase (TH).

22. The isolated cell population of claim 20, wherein said differentiated mesenchymal stem cells express higher levels of Tuj1 than in non-differentiated mesenchymal stem cells.

23. The isolated cell population of claim 20, wherein said differentiated mesenchymal stem cells express neuronal nuclear specific antigen (NeuN).

24. The isolated cell population of claim 20, wherein said differentiated mesenchymal stem cells express Nurr1.

25. The isolated cell population of claim 20, wherein said differentiated mesenchymal stem cells express lower levels of GAD67 than in non-differentiated mesenchymal stem cells.

26. The isolated cell population of claim 20, wherein said mesenchymal stem cells are differentiated in a culture medium comprising brain derived neurotrophic factor (BDNF), wherein said culture medium comprises no more than about 0.01 μM retinoic acid (RA).

27. The isolated cell population of claim 20, wherein said mesenchymal stem cells are differentiated in a culture medium comprising glial-derived neurotrophic factor GDNF, transforming growth factor β3 (TGFβ3) and retinoic acid (RA).

28. A pharmaceutical composition comprising an isolated cell population comprising differentiated mesenchymal stem cells secreting more than 800 pg of dopamine per million cells following KCl depolarization, the cells being non-genetically modified.

29. A method of treating a neurodegenerative disorder in a subject in need thereof, comprising transplanting a therapeutically effective amount of the pharmaceutical composition of claim 28 into the subject, thereby treating the neurodegenerative disorder.

30. The method of claim 29, wherein the neurodegenerative disorder is selected from the group consisting of Parkinson's disease, multiple sclerosis, amyotrophic lateral sclerosis (ALS), autoimmune encephalomyelitis, Alzheimer's disease, Stroke and Huntington's disease.

31. The method of claim 29, wherein the neurodegenerative disorder is Parkinson's disease.
